(19) United States
(12) Patent Application Publication
(10) Pub. No.: US 2013/0177994 A1
(43) Pub. Date: Jul. 11, 2013

(54) METHODS FOR QUANTITATIVE CHIRAL DETERMINATION OF THE d- AND l- ENANTIOMERS OF AMPHETAMINE AND METHAMPHETAMINE

(71) Applicant: Clinical Reference Laboratory, Inc., Lenexa, KS (US)

(72) Inventors: David J. Kuntz, Olathe, KS (US); Michael J. Herrera, Shawnee, KS (US)

(73) Assignee: CLINICAL REFERENCE LABORATORY, INC., Lenexa, KS (US)

(21) Appl. No.: 13/734,468

(22) Filed: Jan. 4, 2013

Related U.S. Application Data
(60) Provisional application No. 61/583,504, filed on Jan. 5, 2012.

(51) Int. Cl. G01N 33/94

(52) U.S. Cl. CPC G01N 33/946
USPC 436/111

(57) ABSTRACT

Methods for the chiral separation and quantitative determination of the d- and l-enantiomers for amphetamine and methamphetamine in bodily fluids and tissues are provided. The method comprises providing a bodily fluid or tissue sample from a subject, extracting target analyte(s) from the sample, followed by eluting on a liquid chromatography column comprising a chiral stationary phase to yield an eluent, which is then analyzed for the presence of the analytes using a mass analyzer.

### CROSS REFERENCE TO RELATED APPLICATIONS

The present application claims the priority benefit of U.S. Provisional Patent Application Ser. No. 61/583,504, filed Jan. 5, 2012, entitled METHOD FOR THE QUANTITATIVE CHIRAL DETERMINATION OF THE d- AND l-ENANTIOMERS OF AMPHETAMINE AND METHAMPHETAMINE, incorporated by reference in its entirety herein.

### BACKGROUND OF THE INVENTION

1. Field of the Invention

The present invention relates to methods with improved accuracy for qualitative and quantitative determination of the d- and l-enantiomers of amphetamines and methamphetamines in bodily fluids and tissues.

2. Description of Related Art

Testing for drug abuse in bodily fluids and tissues has become commonplace. In 2004, the Department of Health and Human Services (DHHS) proposed oral fluid analysis for inclusion into the federal workplace drug testing program. In October of 2011, the Drug Testing Advisory Board (DTAB) for the Substance Abuse and Mental Health Services Administration (SAMHSA) voted to move oral fluid testing forward for inclusion into the federal workplace drug testing program.

Amphetamines and methamphetamines (structures shown in FIG. 1) are commonly tested-for drugs. However, there are several prescription (e.g., Adderall®) and over-the-counter drugs that include or metabolize into amphetamines and/or methamphetamines in the body. This raises issues when interpreting positive drug test results. The testing of bodily fluids and tissues for amphetamines will require laboratories to have the ability to test for the isomers of amphetamine and methamphetamine. That is, amphetamine and methamphetamine enantiomer data must be assessed to differentiate legitimate from illegitimate uses of these substances. For example, although methamphetamines are a controlled substance, the presence of only the l-enantiomer of methamphetamine in a sample indicates the use of a permissible over-the-counter product (Vicks® Vapor Inhaler), whereas the presence of both enantiomers indicates the possible use and/or abuse of a controlled substance. The qualitative and quantitative determination of these enantiomers can identify drug use from an illicit source. However, due to the possible implications of these test results, it is imperative that they be accurate.

The most common gas chromatography/mass spectrometry (GC/MS) method for chiral determination of amphetamine and methamphetamine in urine has been the use of the chiral derivatizing reagent, N-Trifluoroacetyl-L-prolyl chloride (L-TPC), to enable the separation of the d- and l-isomers of amphetamine and methamphetamine. However, it is well known that the TPC reagent is only 98% pure, and degrades over time. Thus, this test has a maximum accuracy of only 98%, and often leads to incorrect false negatives. An alternate GC/MS method using R-(−)-alpha-methoxy(trifluoromethyl)phenylacetyl chloride (MPTA) to prepare amide diastereomers of amphetamine and methamphetamine may also be used to achieve separation of these enantiomers. S-heptafluorobutyrylprolyl chloride can also be used as the derivatization agent for GC/MS along with detection in negative ionization mode. Analytical methods using HPLC and LC/MS have also been reported using other derivatizing agents, such as naphthoyl chloride, with UV or fluorescence detection. Again, these methods relies on reagents which degrade over time and can vary from manufacturer to manufacturer, decreasing their accuracy and reliability.

Thus, there remains a need in the art for reliable and highly accurate methods for the qualitative and quantitative determination of the d- and l-enantiomers of amphetamine and methamphetamine in bodily fluids and tissues.

### SUMMARY OF THE INVENTION

A highly sensitive and specific method of detecting d- and l-enantiomers of amphetamine and/or methamphetamine in a bodily fluid or tissue sample from a subject is provided. The method generally comprises (consists essentially, or even consists of) providing a bodily fluid or tissue sample from the subject and extracting the enantiomers from the sample to yield an extracted sample. The extracted sample is then eluted on a liquid chromatography column comprising a chiral stationary phase to yield an eluent, which is then analyzed for the presence of said enantiomers using a mass analyzer. In one or more embodiments, the sample is extracted using a solid-phase extraction cartridge and the subsequent extract is dried and reconstituted with mobile phase. High performance liquid chromatographic (HPLC) chiral separation is performed, for example, on a Supelco Astec Chirobiotic® V2 column, and detected by a mass analyzer (e.g., spectrometer in the MS/MS mode).

The method is accurate and reproducible for levels as low as about 1 ng/mL to about 200 ng/mL for each enantiomer in the sample. The intra-day (n=6 each day) and inter-day (n=18) reproducibility (CV) for all analytes is less than 6% across the linear range of the method. Preparation and quantitation of spiked 20% d-controls (n=18 over three days) resulted in CV's of less than 2%.

### BRIEF DESCRIPTION OF THE DRAWINGS

FIG. 1 shows the structures of methamphetamine (left) and amphetamine (right);

FIG. 2 is a chromatogram for the calibrator containing 20 ng/mL of each of d- and l-enantiomers of amphetamine and methamphetamine;

FIG. 3 is a chromatogram of the blank oral fluid injection following six consecutive injections of 200 ng/mL standard; and

FIG. 4 is a representative chromatogram of a donor sample.

### DETAILED DESCRIPTION

Described herein are improved methods for the qualitative and quantitative determination of the d- and l-enantiomers of amphetamine and methamphetamine in bodily fluids and tissues using a chiral analytical column. Unlike GC/MS used in previous methods, the use of liquid chromatography mass spectrometry (LC/MS) to analyze the samples, and more specifically LC with a chiral stationary phase column, preferably with tandem mass spectrometry (LC/MS/MS), provides several advantages over existing methods. For example, the present methods rely on a chiral stationary phase and thus eliminate the use of chiral reagents that can degrade over time. Thus, it has been determined that derivatization with a chiral reagent is an unnecessary analytical step for preparing a sample for LC/MS/MS when using an appropriate column containing a chiral stationary phase according to the invention. In some embodiments, the methods exclude any derivatization with a chiral reagent (e.g., N-Trifluoroacetyl-L-prolyl chloride, R-(−)-alpha-methoxy(trifluoromethyl)phenylacetyl chloride, S-heptafluorobutyrylprolyl, and/or naphthoyl chloride) to prepare the sample for analysis. Suitable chiral analytical columns are commercially available, which can simplify the analysis and allow for the separation and quantitation of isomers of drugs. In some embodiments described herein, a macrocyclic antibiotic is used as the chiral stationary phase, although those of skill in the art will recognize that other suitable chiral columns having a different stationary phase may be used to carry out the invention.

In some embodiments, the inventive methods generally comprise providing a biological sample from a subject (e.g., human or non-human mammal). Suitable biological samples will comprise (consist essentially, or even consist of) cells, bodily fluid, tissue, or a combination thereof. It will be appreciated by those in the art that the sample can be collected for use in the invention using any suitable technique (e.g., swabbing, collection cups, etc.), including commercially available kits for collecting such samples (e.g., Intercept® Oral Fluid Drug, Orasure Technologies). The sample can be immediately analyzed after collecting, or the sample can be appropriately stored (e.g., by refrigeration or cooling to temperatures of from about −80° C. to about room temperature (˜25° C.)) for later analysis. Virtually any bodily fluid or tissue that can be collected from a subject can be analyzed using the present invention. Bodily fluids include, without limitation, oral fluids (saliva), sweat, urine, blood, serum, plasma, spinal fluid, and the like. Tissue samples include hair samples (follicle and/or shaft), skin tissue, oral tissue, fat tissue, muscle tissue, and the like. One advantage of the invention is that smaller sample volumes of fluid or tissue can be used for analysis, making it particularly suitable for use with oral fluids and sweat. In some embodiments, the sample size is from about 0.001 to about 1.0 mL, preferably from about 0.050 to about 0.250 mL, and more preferably about 0.200 mL.

The sample can be first diluted in a suitable buffer, although the present invention is also suitable for analyzing neat samples (i.e., samples taken directly from the subject without diluting in a buffer or other processing). In one or more embodiments, internal standard, such as deuterium labeled amphetamine or deuterium labeled methamphetamine, is added to the sample. The target analyte(s) (i.e., isomers/enantiomers of amphetamines and/or methamphetamines) is then extracted from the sample. In one or more embodiments, solid-phase extraction is used for extracting the target analyte. Suitable solid-phase extraction techniques include the steps of conditioning the column with an aqueous or organic solvent, application of the sample to the solid-phase cartridge, washing/rinsing of the cartridge with appropriate aqueous or organic solvents, and eluting of the sample with aqueous or organic solvent to yield an extracted sample. The extracted sample is then dried, and reconstituted into a mobile phase for LC. Drying is typically carried out using evaporation via simple air-drying, vacuum pressure, artificial atmosphere (e.g., nitrogen gas), or a combination thereof. Suitable LC mobile phases for reconstitution are preferably organic solvents, and can comprise conventional HPLC mobile phases such as methanol, ethanol, acetonitrile, isopropanol, and the like. The reconstituted sample will preferably have a volume of from about 0.05 mL to about 0.5 mL, more preferably from about 0.1 mL to about 0.250 mL, and even more preferably about 0.2 mL. The concentration of the extract in the reconstituted sample will preferably be from about 0.1 to about 1000 ng/mL, preferably from about 0.5 to about 500 ng/mL, and more preferably about 20 ng/mL. The reconstituted (and non-derivatized) sample is then injected onto a chiral analytical column for separation on the chiral stationary phase. As noted above, in some embodiments, the chiral stationary phase comprises a macrocyclic antibiotic. Suitable antibiotic stationary phases include, without limitation, vancomycin, β-cyclodextrin, polysaccharide, and the like. The flow rate in the column can be adjusted per the manufacturer's recommendations, but typically ranges from about 0.1 mL/min. to about 1 mL/min., with about 0.5 mL/min. being particularly preferred. The column eluent is then volatized (ionized) and introduced into a suitable mass analyzer for detection and measurement of the target analyte(s). Those skilled in the art will appreciate that ionization can be achieved using any suitable technique (e.g., electrospray, turbospray, photoionization, chemical, thermal, gas, and/or electron), with many suitable ionization machines being commercially available. It will also be appreciated that any suitable mass analyzer can be used in the invention, including, without limitation, a single quadrupole mass spectrometer, triple quadrupole mass spectrometer, ion trap mass spectrometer, time of flight (TCF) mass spectrometer, quadrupole-time of flight (Q-TOF) mass spectrometer, and the like. As noted above, tandem MS mode is particularly suited for some embodiments of the invention. The mass analyzer will generate a mass spectrum for the sample. The results can be compared to positive and/or negative controls and/or other standards known for amphetamine and methamphetamine isomers to determine the drug(s) used by the subject. For example, the generated mass spectrum can be compared with one or more one mass spectrum/spectra stored in a database for amphetamine and/or methamphetamine isomers. Accordingly, the isomers in the sample can be determined based on the comparison between the generated mass spectrum and the database mass spectrum. It will be appreciated that such comparison may be carried out manually or can be automated (computerized). The total run time to detect and measure the target analyte is about 10 minutes or less, as measured from the time of injection onto the column to resolution (detection and measurement) with the mass analyzer. In other words, the method permits sequential analysis of multiple samples, with only about a 10 minute or less waiting period between injections on the column. Those skilled in the art will appreciate that this is much quicker than prior analysis methods.

The method permits determination of respective amounts of d- and/or l-isomers of methamphetamine or amphetamine by comparison of the response for the d- and/or l-isomers of methamphetamine or amphetamine in the fortified or patient sample to a single-point calibrator with the line forced through the origin, which can be reported as ng/mL. A percentage or ratio of d- and/or l-isomers can also be reported. As noted herein, the methods are extremely sensitive and allow the use of a very small sample size of less than about 1 mL, preferably less than about 0.250 mL, and even more preferably about 0.200 mL, while being able to detect analyte levels (amounts) present in the sample as low as 1 ng/mL. The methods are also highly specific with low interference by other compounds, as discussed in the examples below, and achieve “complete” separation of target analytes. As used herein, separation is considered to be acceptable or “complete” when the method achieves a valley-to-peak ratio (aka resolution value) in the mass spectral peaks of about 10% or less, where the valley is measured as the height above the extrapolated baseline at the lowest point of the curve separating the minor and major peaks in the spectrum, and the peak is measured as the height above the extrapolated baseline of the minor peak. Thus, true baseline separation is defined as about 0% resolution value, and methods according to the invention are capable of achieving a resolution value of from about 0% up to about 10%. In one or more embodiments, the methods achieve a resolution value of less than 10%. In other words, these methods have advantageously been shown to have up to about 100% accuracy, which is an important improvement in the state of the art. Thus, the use of a chiral column for the separation of the d- and l-isomer of amphetamine and methamphetamine achieves both quantitative and qualitative accuracy, using a low sample volume of fluid or tissue without the derivatization step required by other methods.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Materials and Methods

### Reagents

Ammonium hydroxide (ACS), Formic acid (96%), o-phosphoric acid (85%), ammonium formate (99.9%), ten-butyl methyl ether (99.8%), and sodium m-periodate were purchased from Sigma-Aldrich (St. Louis, Mo., USA). Glacial acetic acid (ACS grade) was supplied by BDH (VWR, West Chester, Pa., USA). HPLC grade ethyl acetate, isopropyl alcohol, and methanol were supplied by EMD (Philadelphia, Pa., USA). Negative oral fluid was purchased from Orasure Technologies, Inc. (Bethlehem, Pa., USA). The Intercept oral fluid collection device from Orasure Technologies, Inc. was used for donor sample collection. d-amphetamine, 1-amphetamine, d-methamphetamine, l-methamphetamine, D₁₁-amphetamine, and D₁₄-methamphetamine were supplied by Cerilliant (Round Rock, Tex., USA).

### Equipment

Extractions were performed in a 96-well format using Agilent SPEC-DAU, 15 mg, extraction discs (Santa Clara, Calif., USA). Positive pressure was applied using a System 96 multi-channel SPE manifold from SPEWare Corporation (Baldwin Park, Calif., USA). Sample dry down was performed using the SPE Dry-96 from Biotage (Uppsala, Sweden).

### LC/MS/MS Conditions

HPLC conditions were adapted from a separation method posted on the manufacturer's website (Sigma-Aldrich) using UV detection. A Supelco Astec Chirobiotic® V2 chiral column (5 μm particle size, 2.1×250 mm) from Sigma-Aldrich was used to achieve the chiral separation of the enantiomers for amphetamine and methamphetamine. The Chirobiotic® V2 column employs vancomycin as the chiral stationary phase to induce separation. The mobile phase consisted of 99.89:0.1:0.01 methanol:acetic acid:ammonium hydroxide (v/v/v) with a flow rate of 0.5 mL/min. The column temperature was 30° C. The HPLC employed was a Shimadzu Nexera UPLC system (Kyoto, Japan) operating at typical HPLC pressures. The injection volume for the method was 5 μL.

Detection was performed by an API 4000 triple-quadrupole mass spectrometer from AB Sciex (Foster City, Calif., USA). Column eluent was introduced into the mass spectrometer using the Turbolonspray® source operating in the positive ionization mode. Multiple-reaction monitoring (MRM) was used to analyze and detect each compound with a high degree of selectivity. The ionspray voltage was set at 5000V and the source temperature was 550° C. Parameters such as declustering potential (DP), collision energy (CE) and entrance and exit potentials (EP and CXP) were optimized for each analyte. The total analysis time for the method is 10 minutes. The quantitation and qualifier MRM transitions used for the method are listed in Table I below.

TABLE I
Mass Transitions for MS/MS Data Acquisition

| Analyte | Internal Standard | Transitions |
|---|---|---|
| d,l-Amphetamine | | 136.0→91.0 |
| d,l-Amphetamine qualifier | | 136.0→119.1 |
| | D₁₁-Amphetamine | 147.0→98.0 |
| d,l-Methamphetamine | | 150.0→91.1 |
| d,l-Methamphetamine qualifier | | 150.0→119.0 |
| | D₁₄-Methamphetamine | 164.0→98.1 |

### Sample Preparation

Sample extraction was accomplished by aliquoting 200 μL of oral fluid sample into a 16×75 mm culture tube and adding 50 μL of internal standard (200 ng/mL racemic D₁₁-amphetamine and D₁₄-amphetamine). The sample was then treated with 25 μL of 10% sodium periodate solution and allowed to sit for 30 minutes after mixing. A 500 μL aliquot of 0.1 M phosphoric acid was added to each sample and mixed prior to transfer to the SPE wells.

### Solid-Phase Extraction

The Agilent SPEC-DAU 96-well SPE plate was conditioned with 1 mL of methanol followed by 0.5 mL of 0.1 M phosphoric acid. Nitrogen gas was used to apply positive pressure for all SPE steps to induce a flow rate of about 1 to 2 mL per minute. The sample was applied to the SPE well and subsequently rinsed with 300 μL of 0.1 M phosphoric acid. The wells were then washed with 300 μL of 25% isopropyl alcohol in water with 0.2% formic acid followed by a wash of 300 μL of tert-butyl methyl ether. The wells were dried for about 2 minutes using positive pressure and then the samples were eluted with 600 μL of 20:80:2 methanol:ethyl acetate:ammonium hydroxide (v/v/v).

The samples were evaporated to dryness under nitrogen gas at 45° C. and then reconstituted with 200 μL mobile phase. The 96-well block was then centrifuged for 5 minutes at approximately 1800 ref prior to injecting 5 μL on the LC/MS/MS.

Method validation included accuracy and precision, linearity, carryover, recovery, matrix effects, stability and interference. Donor samples were also analyzed to aid in assessing the method.

### Results

### Accuracy and Precision

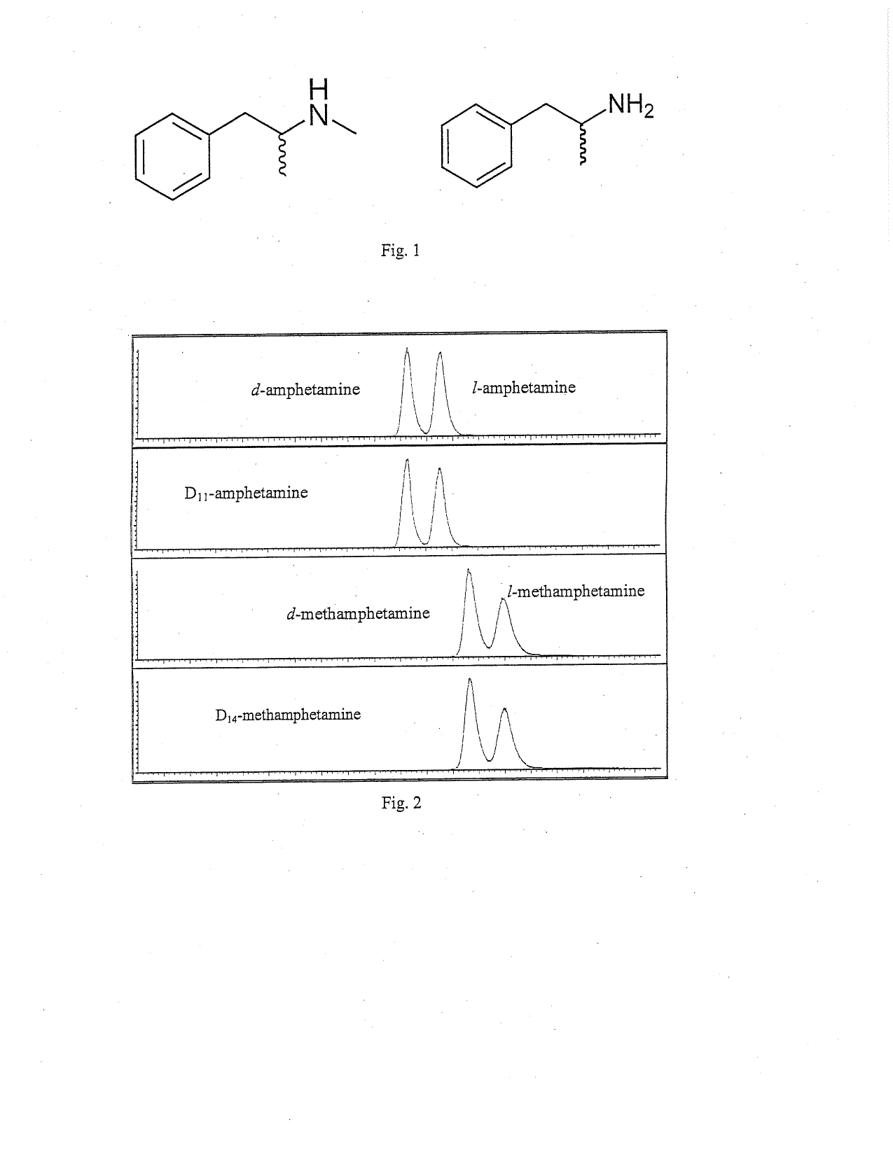

Accuracy and precision across the analytical range were assessed over three days with n=6 replicates each day for a total of n=18. Accuracy and precision were measured at 1.00 ng/mL (limit of quantitation—LOQ), 40 ng/mL, and at 200 ng/mL (upper limit of linearity—ULOL) in oral fluid for each analyte. Quantitation was determined against a single-point calibrator at 20 ng/mL for each analyte (FIG. 2). Intra-day accuracy ranged from −13.5% to +6.9% for all analytes. The inter-day accuracy ranged from −12.0% to 2.8% for all analytes. The only analyte where accuracy was outside of ±10% was d-methamphetamine at 200 ng/mL. Intra-day precision ranged from 0.4% to 5.9%. Intra-day precision values ranged from 1.4% to 5.3%. The results for accuracy and precision are listed in Table II.

TABLE II
Accuracy and Precision Results

| | Intra-day Ranges | | Inter-day Ranges | |
|---|---|---|---|---|
| Analyte | Accuracy | Precision | Accuracy | Precision |
| d-Am- | −9.6% to | 0.4% to | −6.3% to | 1.8% to |
| phetamine | 2.3% | 1.9% | 1.2% | 3.0% |
| l-Am- | −7.8% to | 0.4% to | −4.9% to | 1.4% to |
| phetamine | 3.0% | 3.8% | 1.9% | 3.8% |
| d-Meth- | −13.5% to | 0.3% to | −12.0% to | 1.4% to |
| amphetamine | 0.4% | 2.5% | −0.6% | 2.4% |
| l-Meth- | −7.2% to | 0.9% to | −3.6% to | 3.9% to |
| amphetamine | 6.9% | 5.9% | 2.8% | 5.3% |

Accuracy and precision were also assessed for oral fluid controls containing 20% d-amphetamine and d-methamphetamine and 80% l-amphetamine and l-methamphetamine at total amphetamine and methamphetamine concentrations of 50 ng/mL and 200 ng/mL each (Table III). Intra-day accuracy ranged from −3.7% to +4.8% for all analytes. The inter-day accuracy ranged from −2.2% to 3.9% for all analytes. Intra-day precision ranged from 0.3% to 1.2%. Intra-day precision values ranged from 0.6% to 1.4%.

TABLE III
Accuracy and Precision Results for 20% D Controls

| | Intra-day Ranges | | Inter-day Ranges | |
|---|---|---|---|---|
| Analyte | Accuracy | Precision | Accuracy | Precision |

50 ng/mL Total Amphetamine and Methamphetamine

| | | | | |
|---|---|---|---|---|
| Amphetamine | 2.0% to 2.9% | 0.3% to 0.6% | 2.6% | 0.6% |
| Meth- | −3.4% to −1.1% | 0.4% to 0.8% | −2.0% | 1.2% |
| amphetamine | | | | |

200 ng/mL Total Amphetamine and Methamphetamine

| | | | | |
|---|---|---|---|---|
| Amphetamine | 2.8% to 4.8% | 0.7% to 0.9% | 3.9% | 1.1% |
| Meth- | −3.7% to −1.6% | 0.3% to 1.2% | −2.2% | 1.4% |
| amphetamine | | | | |

### Linearity

Linearity for the quantitation of the d- and l-enantiomers of amphetamine and methamphetamine was assessed from 1 ng/mL to 200 ng/mL using a single-point calibrator of 20 ng/mL. A total of seven concentrations (1, 10, 15, 20, 30, 40, and 200 ng/mL) spread across the range were used to evaluate linearity, n=6 at each concentration. The CV at any given concentration for the n=6 replicates did not exceed 3.5% for any analyte. Linear regression of the mean value determined at each concentration shows an R² value greater than 0.999 for all analytes (Table IV). Based on these results, the linear range of the method is 1 ng/mL to 200 ng/mL.

TABLE IV
Linearity Results for Regression from 1 to 200 ng/mL

| Analyte | Slope | Y-Intercept | R² |
|---|---|---|---|
| d-Amphetamine | 0.8974 | 1.8619 | 0.9996 |
| l-Amphetamine | 0.9037 | 1.7691 | 0.9996 |
| d-Methamphetamine | 0.8554 | 2.6490 | 0.9992 |
| l-Methamphetamine | 0.9714 | 0.5240 | 1.0000 |

### Carryover

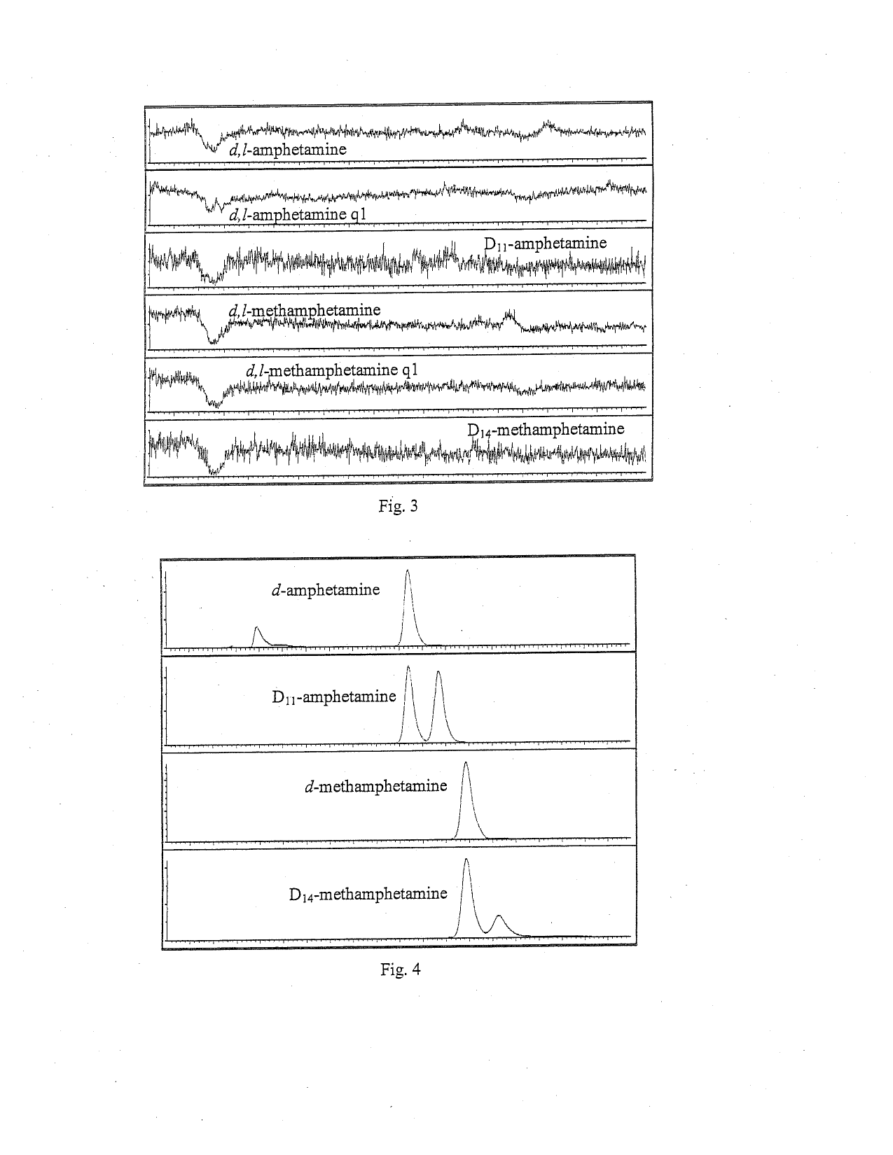

Carryover was assessed by injecting a double blank sample immediately after six consecutive standard injections containing 200 ng/mL each of d- and l-amphetamine and d- and l-methamphetamine. No response was observed in the double blank standard injected immediately after the last injection of the 200 ng/mL standard for d- and l-amphetamine and B- and l-methamphetamine (FIG. 3).

### Recovery

Recovery was assessed at 1 ng/mL and 200 ng/mL for each of d- and l-amphetamine and d- and l-methamphetamine in oral fluid (Table V). Recovery was evaluated by extracting spiked oral fluid samples and comparing peak areas to negative samples spiked post-extraction at equivalent concentrations. Recoveries obtained were greater than 60% for all analytes.

TABLE V
Recovery Results

| | % Recovery | |
|---|---|---|
| Analyte | Analyte | Internal Standard |
| d-Amphetamine | 69 | 67 |
| l-Amphetamine | 68 | 67 |
| d-Methamphetamine | 65 | 62 |
| l-Methamphetamine | 81 | 77 |

### Matrix Effects

The assessment of relative matrix effects on LC/MS/MS analysis in various sources of matrix has been described by Matuszewski (Standard line slopes as a measure of a relative matrix effect in quantitative HPLC-MS bioanalysis. J. Chrom. B. 830: 293-300 (2006)). The ability to obtain accurate results independent of matrix induced ion suppression or enhancement is essential for LC/MS/MS analysis. Matrix effects were assessed in 11 different sources of negative oral fluid collected using the Orasure Intercept Oral Fluid device and spiked at 10 ng/mL for each of d- and l-amphetamine and d- and l-methamphetamine, and compared to a calibration sample prepared in neat oral fluid. Results are presented in Table VI and show that there are no significant adverse relative matrix effects observed on the accuracy and precision of the method. There is also no significant impact on samples collected using the Intercept device when compared to neat oral fluid.

TABLE VI
Matrix Effects Evaluation at 10 ng/mL

| Analyte | # of Sources | Mean % Accuracy | % CV |
|---|---|---|---|
| d-Amphetamine | 11 | 104.5 | 1.0 |
| l-Amphetamine | 11 | 103.2 | 1.1 |
| d-Methamphetamine | 11 | 103.8 | 1.4 |
| l-Methamphetamine | 11 | 101.9 | 1.3 |

### Analyte Stability in Matrix

Stability of amphetamine and methamphetamine in oral fluid was performed by storing the 20 ng/mL standard refrigerated. A fresh standard was prepared at 35 days and compared to the aged standard. Analytes in the aged standard calculated at 102% to 108% of the freshly prepared standard. Stability was demonstrated for amphetamine and methamphetamine in oral fluid for at least 35 days when stored refrigerated.

### Interference Study

An interference study was performed by spiking various drugs into samples containing 20 ng/mL of each of d- and l-amphetamine and d- and l-methamphetamine and also spiking the drugs into negative oral fluid. Table VII lists the compounds evaluated and the concentrations assessed. No significant interference was observed during the study.

TABLE VII
Interference Compounds Assessed

| | Concentration |
|---|---|
| Compounds | (ng/mL) |
| Pseudoephedrine | 125,000 |
| Ephedrine | 100,000 |
| Phenylpropanolamine | 100,000 |
| Phentermine | 10,000 |
| 3,4-methylenedioxyamphetamine (MDA) | 10,000 |
| 3,4-methylenedioxy-N-methylamphetamine (MDMA) | 10,000 |
| 3,4-methylenedioxy-N-ethylamphetamine (MDEA) | 10,000 |
| Phenylephrine | 10,000 |
| Acetominophen | 10,000 |
| Aspirin | 5,000 |
| Chlorpheniramine | 5,000 |
| Caffeine | 5,000 |
| Diphenhydramine | 5,000 |
| Dextromethorphan | 5,000 |
| Ibuprofen | 5,000 |
| Naproxen | 5,000 |

### Discussion

Sufficient accuracy was demonstrated over the analytical range of the method. The method demonstrated a high degree of precision as well even though there was a significant matrix effect observed for the l-methamphetamine. Recoveries above 60% were obtained for all analytes and the internal standards. The deuterated internal standards mirrored the recoveries of the associated analyte which helps to maintain the accuracy and precision of the method.

The ability to accurately and reproducibly quantitate the ratio of d- and l-enantiomers is a very important aspect of this method. The accuracy and precision obtained in the 20% controls both at lower and higher concentration in the method demonstrate that the method is acceptable for use and provides reliable results which can be used to determine possible sources of the drug in the patient.

Initially, the interference study revealed problems with pseudoephedrine causing ion suppression of the d-amphetamine peak. Once this was observed, an oxidation step was added using 10% sodium in-periodate as the oxidizing reagent. This successfully removed the interference from the analysis as no further ion suppression was noted in a follow-up interference study. The validation was then repeated with the oxidation step in place.

Several donor samples have been analyzed by this method. A representative chromatogram is present in FIG. 4. Sample dilution was performed prior to extraction to the approximate level of the calibrator (20 ng/mL) for those samples that were above the upper limit of linearity. The results obtained by this method were comparable to the original results obtained from a non-chiral LC/MS/MS method where samples had been maintained frozen for over one year (Table VIII).

TABLE VIII
Donor Sample Quantitative Results Comparison - LC/MS/MS vs Chiral LC/MS/MS

| | LC/MS/MS (ng/mL) | | Chiral LC/MS/MS (ng/mL) | | % Difference | |
|---|---|---|---|---|---|---|
| Sample | Amphetamine | Methamphetamine | Amphetamine | Methamphetamine | Amphetamine | Methamphetamine |
| 1 | 117 | 375 | 118 | 377 | 1% | 1% |
| 2 | | 428 | 10.8 | 408 | | −5% |
| 3 | 104 | | 136 | | 31% | |
| 4 | 221 | | 213 | | −4% | |
| 5 | 248 | | 289 | | 16% | |
| 6 | | 27 | 12.0 | 32.3 | | 20% |
| 7 | | 431 | 83.4 | 514 | | 19% |
| 8 | | 354 | 45.5 | 431 | | 22% |
| 9 | 132 | | 139 | | 5% | |
| 10 | 189 | | 232 | 7.60 | 23% | |
| 11 | 1220 | | 1386 | 3.02 | 14% | |
| 12 | 429 | | 448 | | 4% | |
| 13 | 132 | | 155 | | 17% | |
| 14 | 361 | | 391 | | 8% | |
| 15 | 112 | 1004 | 111 | 1170 | −1% | 17% |
| 16 | 1192 | | 1705 | | 43% | |
| 17 | | 97 | 47.3 | 121 | | 25% |
| 18 | | 102 | 47.9 | 126 | | 23% |
| 19 | | 98 | 37.3 | 124 | | 27% |
| 20 | 323 | | 336 | | 4% | |
| 21 | | 49 | 14.8 | 61.0 | | 24% |
| 22 | 217 | | 214 | | −1% | |
| 23 | 560 | | 671 | | 20% | |
| 24 | | 341 | 62.1 | 369 | | 8% |
| 25 | | 93 | 19.0 | 96.3 | | 4% |

For those samples containing methamphetamine, all were >90% d-methamphetamine. For samples containing amphetamine, 9 out of 25 contained approximately 70% d-amphetamine which appears to be consistent with Adderall® use. The remaining samples were found to contain 100% d-amphetamine (Table IX).

TABLE IX
Donor Sample Analysis Results

| | Result (ng/mL) | | | |
|---|---|---|---|---|
| | d- | l- | Total | % D |
| Sample | Amphetamine | Amphetamine | Amphetamine | Amphetamine |
| 1 | 118 | 0 | 118 | 100% |
| 2 | 10.8 | 0 | 10.8 | 100% |
| 3 | 94.5 | 41.3 | 136 | 70% |
| 4 | 213 | 0 | 213 | 100% |
| 5 | 197 | 91.6 | 289 | 68% |
| 6 | 12.0 | 0 | 12.0 | 100% |
| 7 | 83.4 | 0 | 83.4 | 100% |
| 8 | 45.5 | 0 | 45.5 | 100% |
| 9 | 139 | 0 | 139 | 100% |
| 10 | 169 | 63.1 | 232 | 73% |
| 11 | 951 | 435 | 1386 | 69% |
| 12 | 325 | 123 | 448 | 73% |
| 13 | 108 | 46.7 | 155 | 70% |
| 14 | 276 | 115 | 391 | 71% |
| 15 | 111 | 0 | 111 | 100% |
| 16 | 1170 | 535 | 1705 | 69% |
| 17 | 47.3 | 0 | 47.3 | 100% |
| 18 | 47.9 | 0 | 47.9 | 100% |
| 19 | 37.3 | 0 | 37.3 | 100% |
| 20 | 240 | 96.2 | 336 | 71% |
| 21 | 14.8 | 0 | 14.8 | 100% |
| 22 | 214 | 0 | 214 | 100% |
| 23 | 671 | 0 | 671 | 100% |
| 24 | 62.1 | 0 | 62.1 | 100% |
| 25 | 19.0 | 0 | 19.0 | 100% |
| | Result (ng/mL) | | | |
| | d- | l- | Total | % D |
| | Metham- | Metham- | Metham- | Metham- |
| Sample | phetamine | phetamine | phetamine | phetamine |
| 1 | 377 | 0 | 377 | 100% |
| 2 | 408 | 0 | 408 | 100% |
| 3 | | | | |
| 4 | | | | |
| 5 | | | | |
| 6 | 32.3 | 0 | 32.3 | 100% |
| 7 | 514 | 0 | 514 | 100% |
| 8 | 431 | 0 | 431 | 100% |
| 9 | | | | |
| 10 | 7.60 | 0 | 7.60 | 100% |
| 11 | 3.02 | 0 | 3.02 | 100% |
| 12 | | | | |
| 13 | | | | |
| 14 | | | | |
| 15 | 1170 | 0 | 1170 | 100% |
| 16 | | | | |
| 17 | 121 | 0 | 121 | 100% |
| 18 | 118 | 7.58 | 126 | 94% |
| 19 | 124 | 0 | 124 | 100% |
| 20 | | | | |
| 21 | 61.0 | 0 | 61 | 100% |
| 22 | | | | |
| 23 | | | | |
| 24 | 369 | 0 | 369 | 100% |
| 25 | 96.3 | 0 | 96.3 | 100% |

### Conclusions

A quantitative chiral LC/MS/MS method has been validated for the determination of the d- and l-enantiomers of amphetamine and methamphetamine. The method is precise and accurate and is currently in use at Clinical Reference Laboratory for donor sample analysis.

We claim:

1. A highly sensitive and specific method of detecting d- and/or l-enantiomers of amphetamine and/or methamphetamine in a biological sample from a subject, said method comprising:
providing a biological sample from said subject;
extracting said enantiomers from said sample to yield an extracted sample;
eluting said extracted sample on a liquid chromatography column comprising a chiral stationary phase to yield an eluent; and
analyzing said eluent for the presence of said enantiomers using a mass analyzer.

2. The method of claim 1, further comprising:
diluting said sample in a buffer prior to said extracting.

3. The method of claim 2, further comprising:
adding internal standard to said diluted sample prior to said extracting.

4. The method of claim 1, further comprising:
drying said extracted sample and reconstituting said extracted sample in an organic solvent prior to said eluting.

5. The method of claim 4, wherein said reconstituted sample comprises from about 0.1 ng/mL to about 1000 ng/mL of said extracted sample.

6. The method of claim 1, wherein said chiral stationary phase comprises a macrocyclic antibiotic.

7. The method of claim 6, wherein said chiral stationary phase comprises vancomycin.

8. The method of claim 1, wherein said providing comprises collecting said sample from said subject.

9. The method of claim 1, wherein said biological sample is a bodily fluid selected from the group consisting of oral fluids (saliva), sweat, urine, blood, serum, plasma, spinal fluid, and combinations thereof.

10. The method of claim 1, wherein said biological sample is tissue selected from the group consisting of hair, skin tissue, oral tissue, fat tissue, muscle tissue, and combinations thereof.

11. The method of claim 1, further comprising storing said biological sample at a temperature of from about −80° C. to about room temperature prior to said extracting.

12. The method of claim 1, wherein said eluent is ionized prior to said analyzing.

13. The method of claim 1, wherein said eluent is ionized via an ionization technique selected from the group consisting of electrospray, turbospray, photo-, chemical, thermal, gas, electron ionization, and combinations thereof.

14. The method of claim 1, wherein said mass analyzer is selected from the group consisting of single quadrupole mass spectrometers, triple quadrupole mass spectrometers, ion trap mass spectrometers, time of flight mass spectrometers, and quadrupole-time of flight mass spectrometers.

15. The method of claim 1, further comprising comparing the presence of said enantiomers after said analyzing to standards known for amphetamine and methamphetamine isomers to determine the drug(s) used by said subject.

16. The method of claim 1, wherein said mass analyzer generates a mass spectrum for said sample, wherein said analyzing comprises comparing said mass spectrum to one or more one mass spectra stored in a database for amphetamine and/or methamphetamine isomers.

17. The method of claim 16, wherein said mass spectrum comprises mass spectral peaks, said peaks corresponding to one of said enantiomers and having a valley-to-peak ratio of about 10% or less.

18. The method of claim 1, wherein said sample is not derivatized with a chiral reagent at any time prior to or during said analyzing.

19. The method of claim 1, wherein said eluting and said analyzing is completed in less than about 10 minutes.

20. The method of claim 1, wherein said method is free from interference from one or more compounds selected from the group consisting of pseudoephedrine, ephedrine, phenylpropanolamine, phentermine, MDA, MDMA, MDEA, phenylephrine, acetominophen, aspirin, chlorpheniramine, caffeine, diphenhydramine, dextromethorphan, ibuprofen, and naproxen.
